(19) United States
(12) Patent Application Publication
(10) Pub. No.: US 2011/0143442 A1
(43) Pub. Date: Jun. 16, 2011

(54) METHOD FOR DETECTING THE PRESENCE OR ABSENCE OF A CHEMICAL SUBSTANCE IN A LIQUID MEDIUM

(75) Inventors: Soubie Alain, Saint Egreve (FR); Jean Brun, Champagnier (FR); Catherine Durand, Saint Theoffrey (FR)

(73) Assignee: COMMISSARIAT A L'ENERGIE ATOMIQUE ET AUX ENERGIES ALTERNATIVES, Paris (FR)

(21) Appl. No.: 12/994,646

(22) PCT Filed: May 26, 2009
(86) PCT No.: PCT/EP2009/056385

§ 371 Date: Mar. 3, 2011

(30) Foreign Application Priority Data
May 27, 2008 (FR) 08/53435

(51) Int. Cl. G01N 33/20; G01N 33/00; G01N 31/22

(52) U.S. Cl. 436/83; 436/101; 422/68.1; 422/400

(57) ABSTRACT

The invention pertains to a method for determining the presence or absence of at least one chemical substance in a liquid medium, comprising a contacting step of said liquid medium with a device comprising at least one substrate coated, in whole or in part on at least one of its sides, with at least one layer comprising material able to be degraded fully or partly by said chemical substance, said material being chosen from among metals, metal alloys, metal oxides.

### TECHNICAL FIELD

The present invention pertains to a method for detecting the presence or absence of a chemical substance in a liquid medium.

This invention can find application in fields in which the chemical substance under consideration is prohibited, the method therefore having a preventive value, or on the contrary in fields in which the chemical substance is expected to be found.

### STATE OF THE PRIOR ART

In the state of the art, there exist a certain number of techniques intended to detect a chemical substance in a liquid medium, notably when the chemical substance is an acid or base.

Therefore it has long been known to have recourse to the pH test strip technique to detect whether a liquid medium contains an acid or a base. This pH paper is impregnated with a universal indicator whose purpose is to change colour gradually in relation to pH. This universal indicator conventionally corresponds to a mixture of coloured indicators, each of the coloured indicators of the mixture having one, even two colour change regions for specifically determined pH ranges. The coloured indicators are chosen so that, by means of the colour change that takes place, it is possible to obtain the pH of a solution rapidly and to cover pH values ranging from 0 to 14.

From a chemical viewpoint, the coloured indicators are organic compounds able to exist in two forms, of which one predominates over the other in relation to pH, each form being capable of selectively absorbing photons at certain wavelengths of the visible spectrum, each absorption wavelength corresponding to an emission wavelength and hence to a colour.

Conventionally, acid-base coloured indicators can be classified into two categories: those derived from triphenylmethane and those derived from azobenzene, the derivatives of triphenylmethane used as indicator possibly being bromothymol blue, bromophenol blue, bromocresol green, cresol red, phenolphtalein, thymolphtaleine, malachite green, whilst the derivatives of azobenzene possibly being helianthin, congo red, methyl red, methyl yellow, alizarin yellow.

While the use of pH paper allows the presence of an acid or base to be detected in a liquid medium, through determining the pH value, it does not however allow the specific determination of the type of acid or base, i.e. in other words it cannot be used to define which type of acid or base is present in said liquid medium.

Some researchers have designed papers impregnated with a coloured indicator capable of specifically recognizing the presence of an acid, as is the case for hydrofluoric acid with paper called <<Fluoride Test Paper>> marketed by Machere-Nager. Before being dipped in solution, this paper has the particularity of being of pink colour but it changes to yellow if the solution comprises a detectable quantity of hydrofluoric acid (e.g. a 1%, 10% and 50% aqueous hydrofluoric acid solution). However, it is found that this paper does not allow the detection of the presence of hydrofluoric acid in the presence of other elements (such as 35% NH₄F), which substantially attenuate the reaction of hydrofluoric acid.

There is therefore a true need for a method to detect a chemical substance in a liquid medium, allowing the detection of this substance over very wide concentration ranges (possibly ranging from a few tenths of a % to over 50%) without the different substances present in the liquid medium being able to hamper the detection of the desired chemical substance.

### DISCLOSURE OF THE INVENTION

The inventors have therefore developed a novel method based on a different principle to the one described above and capable of allowing the detection of a chemical substance over wide ranges of concentration, and without possible hampering of detection by foreign elements.

Therefore, the invention pertains to a method for determining the presence or absence of at least one chemical substance in a liquid medium, comprising a step to contact said liquid medium with a device comprising at least one substrate, fully or partly coated on at least one of its sides with at least one layer comprising a material capable of being degraded in whole or in part by said chemical substance, said material being chosen from among metals, metal alloys, metal oxides (such as Al₂O₃, SiO₂, Fe₂O₃).

It is specified in the meaning of the invention, in the foregoing and in the remainder hereof, that by metal is meant conductive chemical elements e.g. alkali metals, alkaline-earth metals (e.g. Mg), transition metals (e.g. Ni, Ti, Cu, Co and Fe), poor metals (e.g. Al, Ga, In, Sn, Tl, Pb and Bi), metalloid elements (e.g. Si, Ge).

The invention is therefore based on the capacity of some materials to be degraded by certain chemical substances.

As mentioned above, said material able to be degraded can be chosen, in relation to the chemical substance to be detected, from among metals, metal alloys, metal oxides (e.g. SiO₂, Al₂O₃, Fe₂O₃ oxides).

By degradation, is meant the capacity of the above-mentioned materials to lose their physical integrity in the presence of the chemical substance to be detected, this possibly translating as conversion into soluble ions which therefore pass into the liquid medium.

In particular, said material able to be degraded may be a metal or metal alloy.

In this case, degradation conventionally consists of a redox reaction between the metal and the chemical substance (in our case, the chemical substance to be detected) thereby generating metal ions from the metal or metal alloy and hence the dissolution of the latter in the liquid medium. In other words, said metal or metal alloy is degraded into ions that are soluble in said liquid medium, by the chemical substance to be detected. This principle is none other than the principle used for etching some metals with chemical elements.

The above-mentioned layer(s) are advantageously in the form of thin layers deposited on the surface of the substrate, said thin layers conventionally having a thickness ranging from 50 Å to 1 mm, preferably 50 Å to 10 μm. Therefore with said layers, the ratio of the quantity of constituent material of the layer on the substrate surface is particularly low, allowing rapid disappearance of said material (and hence fast detection) by reaction with the chemical substance to be detected if it is present in the liquid medium.

The substrate according to the invention is conventionally a solid substrate possibly in a material chosen from among amorphous ceramic materials such as glass, metal materials or organic materials (e.g. a polymer).

The substrate used as base for detection may have different configurations.

According to a first embodiment, it can be coated on one of its sides with a single layer comprising a material such as defined above, said material able to be degraded by a chemical substance or a mixture of chemical substances present in a liquid medium. In this case, this type of substrate is intended for the detection of a specific chemical substance or a mixture of specific chemical substances.

According to a second embodiment, the substrate can be coated, on one of its sides, with separate regions each consisting of a thin layer of a different material such as defined above, said thin layers possibly being placed side by side or stacked on each other. In this case, this type of substrate is intended for the detection of several chemical substances present in a liquid medium.

Highly diverse substrate configurations can therefore be envisaged, in relation to the chemical substances it is desired to detect in a liquid medium.

Once the contacting step is completed, the detection of the presence or absence of the chemical substance to be detected can be carried out, as is conventional, by a step to visualize the substrate still in contact with said liquid medium or removed therefrom, which is followed by the disappearance or not of all or part of the above-mentioned layer(s), and if disappearance in whole or in part of said layers is ascertained, a step is performed to correlate the disappearance of said layer(s) with the type of chemical substance which generated said disappearance.

This correlation step can be performed quite simply by consulting cross-reference tables which relate a layer in a given material with the chemical substance capable of degrading said layer.

Depending upon the substrate, the visualization step may be conducted in different manners.

According to one variant, the visualization step is performed directly by ascertaining with the naked eye whether the above-mentioned layers have been degraded in full or in part.

This is the case when the substrate is in a transparent material whilst the above-mentioned layer(s) are in an opaque material. The case is the same if both the substrate and said layer(s) have different colours.

If the difference in colour between the substrate and said layer(s) is not sufficiently visible to the naked eye it can be envisaged, between the substrate and said above-mentioned layer(s), to insert a layer that is inert to the substances present in the liquid medium (and notably to the chemical substance to be detected) this layer having a different colour as seen by the naked eye from the layer(s) needed for detection of the chemical substance(s) to be detected. This layer may be of metal type or organic (e.g. a layer of paint) or ceramic (e.g. SiO₂, Si₂N₄).

According to a second variant, the visualization step is performed by ascertaining deformation of the substrate with the naked eye, in the event that one or more chemical substances to be detected are present in the liquid medium.

This is notably the case when the substrate is flexible (having particular regard to its thickness and/or the type of constituent material) and for example when the substrate has a Young's modulus of less than 10 GPa. At the time of depositing the layer or layers capable of being degraded through action of the chemical substance(s) to be detected, internal stresses are stored in said layers, these generating deformation of the substrate on which said layer(s) are deposited. When the substrate coated with said layer(s) is contacted with the liquid medium comprising one or more chemical substances to be detected, the said layer(s) are degraded in whole or in part which leads to full or partial disappearance of the internal stresses stored in said layers, and hence deformation of the substrate that can be seen with the naked'eye. It is therefore possible, by observing the difference in shape of the substrate before contacting and after contacting thereof with said liquid medium, to determine whether the chemical substance(s) are present in the liquid medium.

After visualization, if deduction is not automatic, the operator performs a correlation step to infer from the full or partial degradation of the above-mentioned layer(s) which chemical substance(s) generated this degradation.

This correlation step may include consulting a cross-reference table which relates the detected chemical substance(s) with a given material.

It may not be necessary to carry out a correlation step as indicated above.

This is particularly the case when the substrate comprises identification indices of the substance(s) to be detected, which appear after the contacting of the substrate with the liquid medium, if this medium contains the substance(s) to be detected. These identification indices may materialize as logos, pictograms (e.g. letters or figures) which may come to be seen after full or partial degradation of the layer(s) by the chemical substance(s) to be detected, these indices indicating the presence of said substance(s).

In practice, the above-mentioned layers can be partly coated with a layer of material that is inert to the substance to be detected, this inert layer possibly being deposited so as to form a direct indicator of the substance (this indicator possibly being in the form of the chemical formula of the detected substance). For example, when the substance to be detected is hydrofluoric acid, the inert layer may be in a resin inert to HF and deposited so as to form the acronym HF on one part of the regions able to be degraded by hydrofluoric acid. Therefore the substrate contacted with a hydrofluoric acid solution only leaves an <<HF>> pattern subsisting on the substrate, having the value of an indicator which may be of particular interest for an operator who is not a chemist.

Several possible implementations of this method can be enumerated:
either the operator knows the chemical substance, whose presence or absence it is desired to detect in a liquid medium, in which case the operator will choose the device adapted to the selective detection of this substance;
or the operator wishes to know which chemical substance(s) are present in a liquid medium, in which case the operator will carry out the method using one or more devices such as defined so as to determine the type of substance(s) present in the liquid medium.

Irrespective of the implementation mode, the method may comprise a prior step to prepare the above-mentioned substrates, if these are not available before implementing the method.

This preparation step may conventionally consist of depositing on a substrate the said above-mentioned layer(s), this deposit possibly being made by cathode sputtering.

It is therefore possible, by means of the method of the invention, to access the detection of a very wide range of substances insofar as it is possible, for a given substance to be detected, to prepare the suitable substrate if this is not already available.

The chemical substances which may be detected by the method of the invention can be of various types.

These may be bases, acids or metal elements optionally existing in oxide or halide form.

As examples of metal elements, mention may be made of iron which, in solution, may exist in the form of an iron halide e.g. iron chloride FeCl₃. This substance can be detected when contacted with a substrate containing a layer of aluminium or copper.

Cobalt may also be cited which may exist in oxide form in an aqueous acid solution e.g. a sulphuric acid solution. In said medium, cobalt can be detected when contacted with a substrate comprising a layer of iron, cobalt, copper or nickel.

This method can be used for the detection of acids, and more particularly for the detection of hydrofluoric acid. It may effectively be of particular interest to detect the presence of hydrofluoric acid in sectors in which this acid is especially used, as is the case in microelectronics.

Hydrofluoric acid can be detected by being contacted with a substrate coated with a titanium layer, hydrofluoric acid having the capacity of selectively degrading titanium.

Other acids are able to degrade titanium. It may therefore be necessary, if a substrate coated with a thin layer of titanium shows degradation, to perform additional tests with substrates coated with thin layers of a metal different from titanium e.g. chromium, iron or copper.

If the tests with chromium, iron or copper do not lead to degradation thereof, it can be confirmed that the acid present is hydrofluoric acid.

If, on the other hand, the tests with chromium and iron are conclusive, i.e. they lead to degradation thereof, it can be inferred that the solution does not contain hydrofluoric acid but acids other than hydrofluoric acid such as sulphuric acid.

As examples, mention is made in the table below of those chemical substances which can be detected with the method of the invention using suitable materials deposited on a substrate.

| Chemical substance | Material |
|---|---|
| Solution HCl + HNO₃ | Nickel |
| Solution HF | Ti, Al₂O₃, SiO₂ |
| Solution H₂O₂ + NH₃ | Cu, Ti |
| Solution H₃PO₄ | Al, Mg |
| Solution H₂SO₄ | Cr, Fe, Ti |
| Solution NaOH | Si |
| Solution KOH | Si |
| Solution of oxalic acid | Fe₂O₃ |
| Solution HNO₃ + HF | Ge |
| Solution of isopropyl | Al₂O₃ |
| alcohol + phosphoric acid | |

As mentioned in the foregoing, an operator, after contacting a substrate such as defined above with a liquid medium comprising a substance to be detected, on ascertaining the degradation of at least one metal layer, may be led to referring to a cross-reference table to look up the correlation between the degraded metal layer and the chemical substance which generated this degradation, thereby making it possible to know which chemical substance is present in the liquid medium. It is noted that the rate of disappearance of the layer may be characteristic of the concentration of the chemical substance to be detected. As an example, for a 1% hydrofluoric acid solution, the rate of attack of a thermal oxide is about 60 to 70 Å/mn and for a 10% hydrofluoric acid solution the attack rate of a thermal oxide is 600 to 800 Å/mn.

Therefore, the method further comprises a step to determine the concentration of the detected chemical substance, this determination step possibly comprising measurement of the degradation rate of the layer or layers of material such as defined above.

Therefore, the invention also pertains to a device comprising at least one substrate coated in whole or in part on at least one of its sides with at least one layer comprising a material able to be degraded fully or partly by said chemical substance, said material consisting of a metal, a metal alloy or a metal oxide, and to a detection kit for detecting at least one chemical substance in a liquid medium, comprising:
a device such as defined above; and
an indicator of the detected chemical substance, in the event of degradation of said material.

The indicator may comprise a leaflet to look up the correlation between the material and the detected chemical substance, in the event of degradation of the said material. Therefore said leaflet may comprise a cross-reference table between the material able to be degraded (such as a metal or metal alloy) present on the surface of the substrate, and the detected chemical substance in the event of degradation of said material.

With said kit an operator, through prior consultation of the leaflet, is able to know which chemical substance may be detected and if this is the desired substance to be detected, the appropriate substrate can be used. If the layer or layers of material such as defined previously are degraded after contacting with the liquid medium to be analyzed, the operator can infer the presence of specific chemical substances by looking up the data in the leaflet. If the operator has no idea which substance is to be detected in a liquid medium, several substrates can be tested and by consulting the leaflet it can be determined which composition is contained in the liquid medium.

The invention is now described in more detail with reference to the examples given below for the purpose of illustration and which are non-limiting.

### BRIEF DESCRIPTION OF THE DRAWINGS

FIG. 1A shows an overhead view of a substrate which, on one of its sides, comprises 5 separate regions each consisting of a different thin metal layer before being placed in contact with a liquid medium to implement Example 1, and FIG. 1B is an overhead view of the same substrate after contacting with the liquid medium.

FIG. 2A is a side view of a substrate coated with a stack of 5 thin metal layers before being placed in contact with a liquid medium, to implement Example 2, and FIG. 2B is a side view of the same substrate after contacting with the liquid medium.

FIG. 3A gives a side view and an overhead view of a substrate coated with a titanium layer and having an indicator, before it is placed in contact with a liquid medium to implement Example 3, and Example 3B is a side view and an overhead view of the same substrate after contacting with the liquid medium.

### DETAILED DESCRIPTION OF PARTICULAR EMBODIMENTS

### Example 1

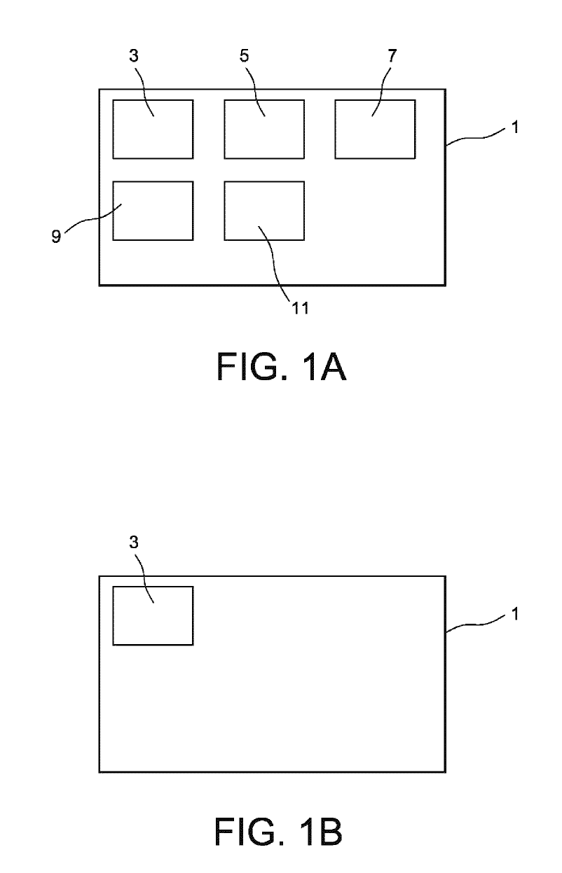

On a substrate 1 of rectangular shape, five rectangular regions 3, 5, 7, 9 and 11 are deposited respectively comprising a thin aluminium layer, a thin copper layer, a thin cobalt layer, a thin nickel layer and a thin iron layer, as illustrated on FIG. 1A. This substrate is immersed in an aqueous solution containing both CrO₃ and sulphuric acid.

This leads to disappearance of the layers of copper, cobalt, nickel and iron. Only region 3 subsists consisting of a thin aluminium layer, indicating the presence of chromium in a sulphuric medium (FIG. 1B).

### Example 2

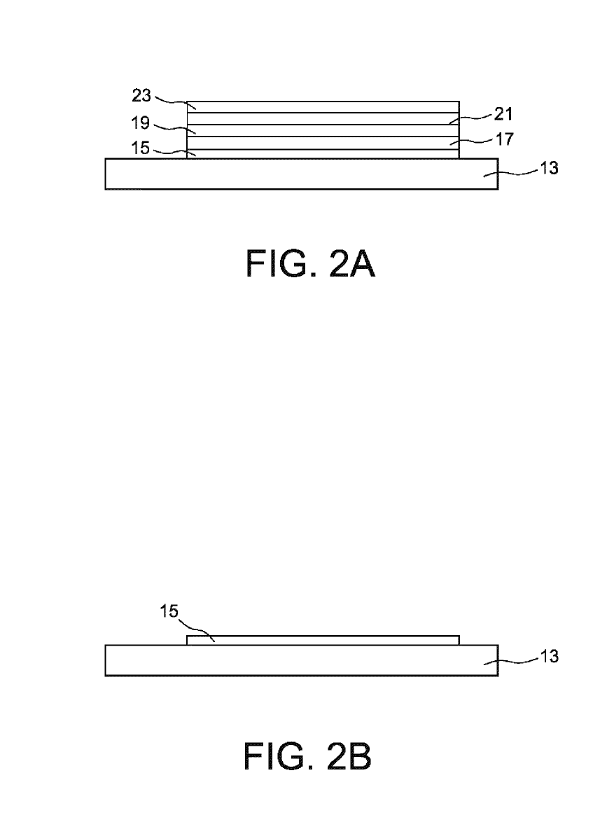

This example is a variant of Example 1, in which the rectangular regions 15, 17, 19, 21 and 23 respectively consist of thin layers of aluminium, nickel, cobalt, iron and copper which are stacked one on the other on a substrate 13, as illustrated on FIG. 2A.

The substrate 13 is then immersed in an aqueous solution containing both CrO₃ and sulphuric acid. Only region 15 continues to subsist which is the thin aluminium layer, indicating the presence of chromium in a sulphuric medium (FIG. 2B).

### Example 3

This example illustrates the detection of hydrofluoric acid in an aqueous solution.

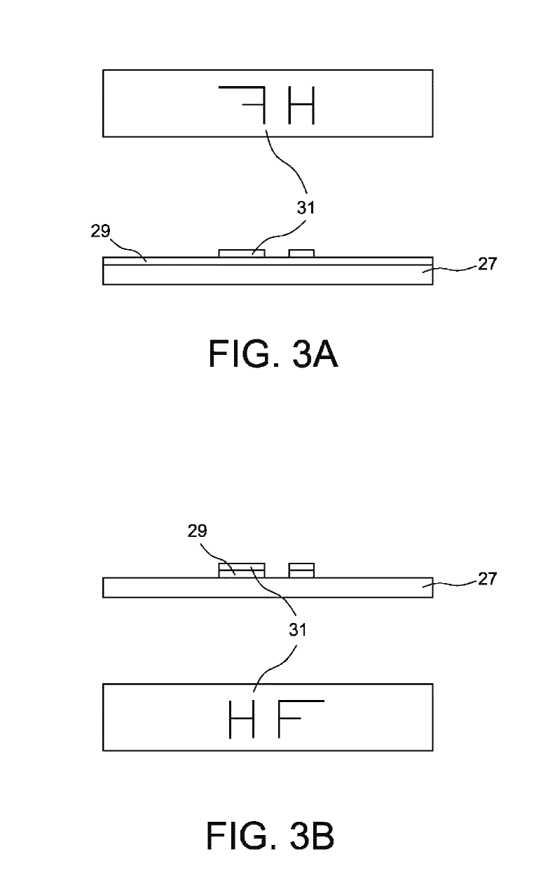

For this purpose, a substrate 27 in glass or transparent polymer having a thickness possibly ranging from 100 μm to 1 mm is coated with a titanium layer 29 of thickness 300 to 1000 Å, this titanium layer being partly coated with a layer of resin 31 that is inert to hydrofluoric acid, this resin layer forming the acronym <<HF>> on the titanium layer (FIG. 3A overhead view). This resin layer is deposited by photolithography.

The substrate is then immersed in a solution comprising hydrofluoric acid. After a few minutes, the titanium layer is seen to disappear, apart from the areas coated with said resin, leaving the resin layer 31 exposed forming the acronym HF (FIG. 3B overhead view)

1. Method for determining the presence or absence of at least one chemical substance in a liquid medium, comprising a contacting step of said liquid medium with a device comprising at least one substrate coated, in whole or in part on at least one of its sides, with at least one layer comprising a material able to be fully or partly degraded by said chemical substance, said material being chosen from among metals, metal alloys, metal oxides.

2. The method according to claim 1, wherein said material able to be degraded is a metal, a metal alloy or a metal oxide which degrades into soluble ions in the presence of said chemical substance to be detected in said liquid medium.

3. The method according to claim 1, further comprising a step to visualize the substrate, still in contact with said liquid medium or removed therefrom, to ascertain the disappearance or not of all or part of the layers mentioned in claim 1.

4. The method according to claim 3 which, after the visualization step, comprises a correlation step between the degradation of said layer(s) and the type of chemical substance which generated said degradation.

5. The method according to claim 1, wherein said layer is a thin layer, said thin layer having a thickness ranging from 50 Å to 1 mm, preferably 50 Å to 10 μm.

6. The method according to claim 1, wherein the substrate is in an amorphous ceramic material.

7. The method according to claim 1, wherein the substrate is coated on one of its sides with a single layer comprising a material according to any of the preceding claims.

8. The method according to claim 1, wherein the substrate is coated on one of its sides with separate regions each consisting of a thin layer of different material.

9. The method according to claim 8, wherein the separate regions lie side by side or are stacked on one another.

10. The method according to claim 1, wherein the substrate comprises identification indices of the substance(s) to be detected, in the form of logos or pictograms, which appear after full or partial degradation of the layer(s) by the chemical substance(s) to be detected, these indices indicating the presence of said substance(s).

11. The method according to claim 1, comprising a prior step to prepare the above-mentioned substrates.

12. The method according to claim 1, wherein the substance to be detected is a base, an acid, a metal element possibly existing in oxide or halide form.

13. The method according to claim 1, wherein the substance to be detected is hydrofluoric acid and the substrate is coated with a layer of titanium.

14. The method according to claim 1, further comprising a step to determine the concentration of the detected chemical substance.

15. The method according to claim 14, wherein the step to determine the concentration of the detected chemical substance comprises measurement of the rate of degradation of the layer(s) of material.

16. Device to implement a method defined according to claim 1, comprising at least one substrate coated in whole or in part on at least one of its sides with at least one layer comprising a material able to be degraded fully or partly by said chemical substance, said material selected from the group consisting of a metal, a metal alloy and a metal oxide.

17. Detection kit to detect at least one chemical substance in a liquid medium, comprising:
a device such as defined in claim 16; and
an indicator of the detected chemical substance, in the event of degradation of said material.

18. Detection kit according to claim 17, wherein the indicator consists of a leaflet cross-referencing the relationship between the material and the detected chemical substance, in the event of degradation of said material.
